(19) United States
(12) Patent Application Publication
(10) Pub. No.: US 2008/0299585 A1
(43) Pub. Date: Dec. 4, 2008

(54) METHODS FOR DETECTION AND MEASUREMENT OF SECRETORY PHOSPHOLIPASE A2 LEVELS (sPLA2) IN BIOLOGICAL FLUIDS

(76) Inventors: Paul Truex, Pleasanton, CA (US); Joaquim Trias, Millbrae, CA (US)

Correspondence Address: PERKINS COIE LLP, POST OFFICE BOX 1208, SEATTLE, WA, 98111-1208, US

(21) Appl. No.: 11/958,238

(22) Filed: Dec. 17, 2007

Related U.S. Application Data
(60) Provisional application No. 60/875,694, filed on Dec. 18, 2006.

(51) Int. Cl. G01N 33/573

(52) U.S. Cl. 435/7.4

(57) ABSTRACT

Elevated levels of secretory phospholipase A₂ (sPLA₂) are associated with a variety of inflammatory conditions, e.g., multiple sclerosis, arteriosclerosis, rheumatoid arthritis, osteoarthritis and sickle cell. ELISA-based assays have been developed for detecting and measuring sPLA₂ levels in biological fluids, but these methods are too time-consuming for practical clinical diagnostic use. Disclosed herein in certain embodiments are methods for rapid detection and measurement of sPLA₂ levels (e.g., sPLA₂ type IIA) in a biological fluid generally, and methods for detection and measurement of sPLA₂ levels in urine.

### CROSS-REFERENCE TO RELATED APPLICATION

The present application claims priority to U.S. Provisional Patent Application No. 60/875,694, filed Dec. 18, 2006, the disclosure of which is incorporated by reference herein in its entirety, including drawings.

### BACKGROUND

Phospholipases A₂ (PLA₂) are a superfamily of enzymes that hydrolyze the ester bond at the sn-2 position of phosphoglycerides to release free fatty acid and lysophospholipids. The superfamily is divided into three groups. One of these groups, secretory phospholipase A₂ (sPLA₂), includes small enzymes of around 14 kDa that requires millimolar concentrations of Ca²⁺ to function.

sPLA₂ catalyzes the release of arachidonic acid from phospholipids during the inflammatory response. This in turn stimulates the production of leukotrienes, prostacyclins, and other inflammation mediators. Elevated levels of sPLA₂ (also known as synovial fluid PLA₂) have been correlated to a variety of inflammatory conditions, such as for example atherosclerosis (Hurt-Camejo 2001), coronary artery disease (Boekholdt 2005), multiple sclerosis (Cunningham 2006), Alzheimer's disease (Moses 2006), sickle cell disease (Styles 1996), and rheumatoid arthritis and osteoarthritis (Jamal 1998).

Several enzyme-linked immunosorbent assay (ELISA) based methods have been developed for detecting and measuring sPLA₂. However, these methods are relatively time-consuming, making them impractical for clinical use in the rapid diagnosis of sPLA₂-related conditions. In addition, these methods are not effective for measuring sPLA₂ levels in certain biological fluids such as urine. Therefore, there is a need for improved methods of detecting and measuring sPLA₂.

### SUMMARY

In certain embodiments, methods are provided for rapidly detecting and/or measuring sPLA₂ levels in a biological fluid sample from a subject using a modified ELISA immunoassay technique. In this assay, the biological fluid sample is applied to a well pre-coated with capture antibody that binds sPLA₂, followed by addition of an acetylcholinesterase conjugate that binds a sPLA₂ at a different epitope than the capture antibody. In certain embodiments, the capture antibody and conjugate bind to sPLA₂ type IIA. In certain embodiments, the biological fluid sample may be serum or urine, and the sample may be diluted prior to loading onto the plate. Following an incubation period of 15 to 60 minutes at 15 to 30° C., the well is washed, Ellman's reagent is added, and the plate is developed for 15 to 25 minutes at 15 to 30° C. In certain embodiments, the incubation period is 15 to 25 minutes. The absorbance of the developed sample is measured at 400 to 412 nm, and the sPLA₂ concentration of the sample is determined by comparing this absorbance reading to a standard curve that plots absorbance versus sPLA₂ concentration for a set of standards with known sPLA₂ concentrations. In certain embodiments, the standard samples that are used to generate the standard curve may be samples that were run at the same time as the test samples. In other embodiments, the absorbances of the standard samples were obtained in a previous assay. In certain embodiments, one or more quality control samples containing known concentrations of sPLA₂ may be tested at the same time as the test samples. In these embodiments, results obtained for the quality control samples may be used to verify the accuracy of the standard curve or the viability of the assay reagents. In certain embodiments, this rapid sPLA₂ measuring method has a total running time of about 120 minutes or less, with certain of these embodiments having a total running time of about 100 minutes or less or about 80 minutes or less.

In certain embodiments, methods are provided for detecting and/or measuring sPLA₂ levels in a urine sample from a subject using a modified ELISA immunoassay technique. In this assay, the urine sample is applied to a well pre-coated with capture antibody that binds sPLA₂, followed by addition of an acetylcholinesterase conjugate that binds a sPLA₂ at a different epitope than the capture antibody. In certain embodiments, the capture antibody and conjugate bind to sPLA₂ type IIA. In certain embodiments, the sample may be diluted prior to loading onto the plate. Following an incubation period of 15 minutes to 24 hours at 15 to 30° C., the well is washed, ElIman's reagent is added, and the plate is developed for 30 minutes to 24 hours at 15 to 30° C. In certain embodiments, the development time is 30 to 90 minutes. The absorbance of the developed sample is measured at 400 to 412 nm, and the sPLA₂ concentration of the sample is determined by comparing this absorbance reading to a standard curve that plots absorbance versus sPLA₂ concentration for a set of standards with known sPLA₂ concentrations. In certain embodiments, the standard samples that are used to generate the standard curve may be samples that were run at the same time as the test samples. In other embodiments, the absorbances of the standard samples were obtained in a previous assay. In certain embodiments, one or more quality control samples containing known concentrations of sPLA₂ may be tested at the same time as the test samples. In these embodiments, results obtained for the quality control samples may be used to verify the accuracy of the standard curve or the viability of the assay reagents.

In certain embodiments, methods are provided for rapidly determining whether the sPLA₂ level in a biological fluid sample from a subject is at or above a threshold concentration using a modified ELISA immunoassay technique. In this assay, the biological fluid sample is applied to a well pre-coated with capture antibody that binds sPLA₂, followed by addition of an acetylcholinesterase conjugate that binds a sPLA₂ at a different epitope than the capture antibody. In certain embodiments, the capture antibody and conjugate bind to sPLA₂ type IIA. In certain embodiments, the biological fluid sample may be serum or urine, and the sample may be diluted prior to loading onto the plate. Following an incubation period of 15 to 60 minutes at 15 to 30° C., the well is washed, ElIman's reagent is added, and the plate is developed for 15 to 25 minutes at 15 to 30° C. In certain embodiments, the incubation period is 15 to 25 minutes. The absorbance of the developed sample is measured at 400 to 412 nm, and this absorbance reading is compared to the average absorbance reading of one or more standard samples having an sPLA₂ concentration that is equal to the threshold concentration. In certain embodiments, the absorbance reading of the test sample is also compared to the average absorbance reading for one or more standard samples having sPLA₂ concentrations above or below the threshold concentration. In certain embodiments, the test sample is classified as having an sPLA₂ concentration at or above the threshold concentration if it has an absorbance reading that is at least 80% of the average absorbance reading for the one or more standard samples having an sPLA₂ concentration equal to the threshold concentration. In other embodiments, the test sample is classified as having an sPLA₂ concentration at or above the threshold concentration if it has an absorbance reading that is at least 90% of the average absorbance reading for the one or more standard samples having an sPLA₂ concentration equal to the threshold concentration, and in still other embodiments it is classified as above the threshold concentration if it has an absorbance reading that is at least 100% that of the average for the standard samples. In certain embodiments, the standard samples may be samples that were run at the same time as the test samples. In other embodiments, the absorbances of the standard samples may have been obtained in a previous assay. In certain embodiments, one or more quality control samples containing known concentrations of sPLA₂ may be tested at the same time as the test samples. In these embodiments, results obtained for the quality control samples may be used to verify the accuracy of the results or the viability of the assay reagents.

In certain embodiments, methods are provided for determining whether the sPLA₂ level in a urine sample from a subject is at or above a threshold concentration using a modified ELISA immunoassay technique. In this assay, the urine sample is applied to a well pre-coated with capture antibody that binds sPLA₂, followed by addition of an acetylcholinesterase conjugate that binds a sPLA₂ at a different epitope than the capture antibody. In certain embodiments, the capture antibody and conjugate bind to sPLA₂ type IIA. In certain embodiments, the sample may be diluted prior to loading onto the plate. Following an incubation period of 15 minutes to 24 hours at 15 to 30° C., the well is washed, Ellman's reagent is added, and the plate is developed for 30 minutes to 24 hours at 15 to 30° C. In certain embodiments, the development period is 30 minutes to 90 minutes. The absorbance of the developed sample is measured at 400 to 412 nm, and this absorbance reading is compared to the average absorbance reading of one or more standard samples having an sPLA₂ concentration that is equal to the threshold concentration. In certain embodiments, the absorbance reading of the test sample is also compared to the average absorbance reading for one or more standard samples having sPLA₂ concentrations above or below the threshold concentration. The test sample is classified as having an sPLA₂ concentration at or above the threshold concentration if it has an absorbance reading that is at least 80% of the average absorbance reading for the one or more standard samples having an sPLA₂ concentration equal to the threshold concentration. In other embodiments, the test sample is classified as having an sPLA₂ concentration at or above the threshold concentration if it has an absorbance reading that is at least 90% of the average absorbance reading for the one or more standard samples having an sPLA₂ concentration equal to the threshold concentration, and in still other embodiments it is classified as above the threshold concentration if it has an absorbance reading that is at least 100% that of the average for the standard samples. In certain embodiments, the standard samples may be samples that were run at the same time as the test samples. In other embodiments, the absorbances of the standard samples may have been obtained in a previous assay. In certain embodiments, one or more quality control samples containing known concentrations of sPLA₂ may be tested at the same time as the test samples. In these embodiments, results obtained for the quality control samples may be used to verify the accuracy of the results or the viability of the assay reagents.

In certain embodiments, methods are provided for detecting or measuring sPLA₂ levels in a biological fluid sample from a subject using a strip assay. In this assay, the biological fluid sample is applied to a strip with a surface comprising capture antibody that binds sPLA₂. In certain embodiments, the biological fluid sample may be serum or urine, and the sample may be diluted prior to applying the sample to the strip. An acetylcholinesterase conjugate that binds a sPLA₂ at a different epitope than the capture antibody is then applied to the strip. In certain embodiments, the capture antibody and conjugate bind to sPLA₂ type IIA. The strip is incubated for 15 minutes to 24 hours at 15 to 30° C. The strip is then optionally washed, Ellman's reagent is applied to the strip, and it is developed for 15 minutes to 24 hours at 15 to 30° C. In certain embodiments, the incubation period is 15 to 60 minutes, and in certain of these embodiments, the incubation period is 15 to 25 minutes. In certain embodiments, the development period is 15 to 90 minutes, and in certain of these embodiments, the incubation period is 30 to 90 minutes. Following development, the color and/or color intensity of the strip is observed and compared to the color and/or color intensity of a standard strip to which a standard sample of known sPLA₂ concentration was applied. In certain embodiments, the test sample is considered to have an sPLA₂ concentration at or above a threshold level if the color and/or intensity of the strip is the same that of a strip treated with a standard sample that has an sPLA₂ concentration at or above the threshold level.

In certain embodiments, the methods disclosed herein may be used to diagnose a condition associated with elevated sPLA₂ levels. Therefore, in certain embodiments, methods are provided for diagnosing a condition associated with elevated sPLA₂ levels using a modified ELISA immunoassay. In these embodiments, a biological fluid test sample from a subject is applied to a well pre-coated with capture antibody that binds sPLA₂, followed by addition of an acetylcholinesterase conjugate that binds a sPLA₂ at a different epitope than the capture antibody. In certain embodiments, the capture antibody and conjugate bind to sPLA₂ type IIA. In certain embodiments, the sample may be diluted prior to loading onto the plate. Following an incubation period of 15 minutes to 24 hours at 15 to 30° C., the well is washed, ElIman's reagent is added, and the plate is developed for 15 minutes to 24 hours at 15 to 30° C. In certain embodiments, the incubation period is 15 to 60 minutes, and in certain of these embodiments, the incubation period is 15 to 25 minutes. In certain embodiments, the development period is 15 to 90 minutes. In certain of these embodiments the development period is 15 to 25 minutes, while in other embodiments it is 30 to 90 minutes. The absorbance of the developed sample is measured at 400 to 412 nm. In certain embodiments, this absorbance reading is used to determine the concentration of the test sample by comparing the absorbance reading to a standard curve that plots absorbance versus sPLA₂ concentration for a set of standards with known sPLA₂ concentrations. In these embodiments, the subject is classified as having a condition associated with elevated sPLA₂ levels if the test sample has an sPLA₂ concentration of 25 ng/ml or greater. In certain of these embodiments, the subject is classified as having a condition associated with elevated sPLA₂ levels if the test sample has an sPLA₂ concentration of 50 ng/ml, in other embodiments 75 ng/ml or greater, and in still other embodiments 100 ng/ml or greater. In other embodiments, the absorbance of the test sample is compared to the average absorbance reading for one or more standard samples having an sPLA₂ concentration of 25 ng/ml or greater. In certain of these embodiments, the standard samples have an sPLA₂ concentration of 50 ng/ml or greater, in other embodiments 75 ng/ml or greater, and in still other embodiments 100 ng/ml or greater. The subject is classified as having a condition associated with elevated sPLA₂ levels if the test sample has an absorbance reading that is at least 90% of the average absorbance reading for the one or more standard samples. In certain embodiments, the standard samples may be samples that were run at the same time as the test samples. In other embodiments, the absorbances of the standard samples may have been obtained in a previous assay. In certain embodiments, one or more quality control samples containing known concentrations of sPLA₂ may be tested at the same time as the test samples. In these embodiments, results obtained for the quality control samples may be used to verify the accuracy of the results or the viability of the assay reagents. In certain of these embodiments, the condition associated with elevated sPLA2 levels is a condition associated with inflammation, such as for example atherosclerosis, coronary artery disease, multiple sclerosis, Alzheimer's disease, sickle cell disease, rheumatoid arthritis, or osteoarthritis.

In certain embodiments, kits are provided for performing the ELISA immunoassay techniques disclosed herein. In certain of these embodiments, the kit may comprise a standard curve and/or a document correlating specific absorbance readings to specific sPLA₂ concentrations.

### BRIEF DESCRIPTION OF THE DRAWINGS

FIG. 1: Effect of alterations in incubation time and incubation temperature on assay sensitivity.

FIG. 2: Effect of alterations in conjugate concentration and incubation temperature on assay sensitivity.

FIG. 3: Effect of alterations in conjugate concentration and development time on assay sensitivity.

FIG. 4: Standard curves generated for buffer and spiked urine samples.

FIG. 5: Standard curves generated for buffer and spiked urine samples.

FIG. 6: Standard curves generated for buffer and spiked urine samples.

FIG. 7: Stability of EIA plate through three freeze/thaw cycles.

### DETAILED DESCRIPTION

The following description of the invention is intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The phrase “biological fluid” as used herein refers to any fluid of human or animal origin, including but not limited to serum, urine, blood, blood plasma, saliva, tears, mucus, and lymph fluid. In certain preferred embodiments, the biological fluid is serum or urine.

“Subject” as used herein refers to any mammal, preferably human.

Methods are provided herein for measurement of sPLA₂ levels in various biological fluids, including serum and urine, using an EIA double-antibody sandwich technique. These methods were developed using materials from a commercially available sPLA₂ (human type IIA) EIA (enzyme immunoassay) kit (Cayman Chemical Co., Ann Arbor, Mich., Catalog No. 585000) (“the Cayman kit”). In certain embodiments, the methods disclosed herein may be carried out using materials from the Cayman kit (e.g., buffers, plate, etc.). In other embodiments, the methods may be carried out using equivalent materials, such as for example materials from a different kit. Such equivalent materials will be readily apparent to one of ordinary skill in the art.

In the Cayman kit, EIA buffer is prepared by diluting the contents of one vial of EIA Buffer concentrate with 90 ml of deionized water free of trace organic contaminants (UltraPure). The vial is rinsed to remove any salts that may have precipitated. Wash buffer is prepared by diluting the contents of a 5 ml vial of Wash Buffer Concentrate to a total volume of 2 liters with UltraPure water and adding 1 ml of Tween 20. Wash buffer is also prepared by diluting the contents of a 12.5 ml vial of Wash Buffer Concentrate to a total volume of 5 liters with UltraPure water and adding 2.5 ml of Tween 20. A smaller volume of Wash Buffer can be prepared by diluting the Wash Buffer Concentrate 1:400 and adding Tween 20 (0.5 ml/L of Wash Buffer). The sPLA₂ standard is reconstituted in 2 ml EIA Buffer. The concentration of this solution is 10 ng/ml, and it is stored at 4° C. for use within one week. Acetylcholinesterase:Fab′ conjugate (AChE:Fab′) (“conjugate”) (100 dtn) is reconstituted with 10 ml of EIA Buffer. 500 dtn conjugate is reconstituted with 50 ml of EIA Buffer. Reconstituted conjugate may be stored at 4° C. for use within six weeks.

The Cayman kit is designed for measuring sPLA₂ levels, and has been validated for use with plasma or synovial fluid only. Measuring sPLA₂ levels in a sample using the Cayman kit is time consuming, because the kit requires an overnight incubation period. Test samples can generally be assayed with no prior purification. The Cayman kit recommends that plasma samples be diluted at least 1:20 and synovial fluid samples should be diluted at least 1:1000. Non-specific mouse serum may be added to test samples in certain cases to compensate for interference from human anti-mouse IgG heterophilic antibodies. If mouse serum is used, it is first reconstituted with 2.5 ml (100 dtn) or 12.5 ml (500 dtn) UltraPure water. 25 μl of reconstituted mouse serum is added to every 500 μl of test or control sample.

A 96-well EIA plate is supplied with the Cayman kit. Each well of this EIA plate is pre-coated with a monoclonal sPLA₂ antibody (“capture antibody”). The capture antibody specifically binds any sPLA₂ type IIa protein, but does not cross-react with type I or type IV PLA₂. The capture antibody also does not appear to cross-react with type V PLA₂. Eight sPLA2 standard samples are added in duplicate at 100 μl/well to the 16 standard wells. Test samples are added to the plate at 100 μl/well, and each sample is assayed in triplicate. Conjugate is added 100 μl/well to each well except for eight blank wells. The conjugate binds a different sPLA₂ type IIA epitope than the capture antibody, allowing the capture antibody and the conjugate to form a “sandwich.” This sandwich is immobilized on the plate, and excess reagents are washed away. The plate is covered with plastic film and incubated overnight at 4° C.

Prior to development, the wells are washed five to six times with wash buffer. To develop the plate, 100 dtn ElIman's Reagent is reconstituted with 20 ml of UltraPure water (or 250 dtn stock is reconstituted with 50 ml UltraPure water), and 200 μl reconstituted reagent is added to each well. The reconstituted Ellman's Reagent must be used the same day it is prepared because it is unstable. ElIman's Reagent contains the AChE substrate. Specifically, Ellman's Reagent contains acetylthiocholine and 5,5′-dithio-bis-(2-nitrobenzoic acid). Hydrolysis of acetylthiocholine by acetylcholinesterase produces thiocholine, which reacts with 5,5′-dithio-bis-(2-nitrobenzoic acid) to produce 5-thio-2-nitrobenzoic acid. 5-thio-2-nitrobenzoic acid has a strong absorbance at 412 nm. The intensity of absorbance at this wavelength is directly proportional to the concentration of bound conjugate, which is in turn is directly proportional to the concentration of sPLA₂ present (Absorbance a [AChE:Fab′ conjugate]α[sPLA₂]).

During the development step, the plate is shaken in the dark. The plate may be checked periodically over the next few hours. Unlike other enzymes commonly used in enzyme immunoassays such as horseradish peroxidase, AChE does not auto-inactivate during turnover. This means that the plate may be read at any time without stopping the reaction, and may be read multiple times. Once the standard wells are visibly yellow (detection of an absorbance of at least 0.3 A.U.), which usually occurs within 20-30 minutes, absorbance values for the various samples may be obtained. Longer development times are necessary to obtain an accurate plot for the lower range of the standard curve and statistically significant values for sample concentrations near the detection limit of the assay. The plate is transferred to a plate reader set at an absorbance value of 412 nm and absorbance readings are obtained for each well. A standard curve is generated by plotting absorbance at 412 nm on the Y-axis and sPLA₂ concentration for standard samples on the X-axis and constructing a best-fit line through the points.

To develop methods for more rapid measurement of sPLA₂ in biological fluids, various combinations of variables were adjusted to determine optimal assay conditions. The first variables to be analyzed were incubation time and temperature. Ideally, a rapid detection assay for use in a clinical setting can be carried out at room temperature (approximately 15-30° C.). Human serum control samples were incubated for 30, 45, 60, or 90 minutes at 4° C., 25° C., or 37° C. Although the absorbance readings of the samples at 405 nm increased with increasing incubation time and temperature, it was found that an incubation time as short as 30 minutes at room temperature (25° C.) resulted in an absorbance reading within the target range of 0.5 to 1.6 AU.

The next variables to be analyzed were incubation time, development time, and conjugate concentration. Human serum control samples were incubated for 30, 45, 60, or 90 minutes in the presence of 2× or 4× conjugate (2× and 4× refer to double or quadruple the concentration of the conjugate in the Cayman kit), and developed for 5, 10, 20, or 90 minutes. Although the absorbance readings at 405 nm increased with increasing incubation time, development time, and conjugate concentration, target absorbance values were still obtained with a 30 minute incubation and 20 minute development at 4× conjugate, or with a 60 minute incubation and 20 minute development at 2× conjugate. Thus, increasing the conjugate concentration greatly decreases the incubation and development times necessary to obtain accurate results.

The third set of variables to be examined were conjugate concentration and temperature. As stated above, it had been shown that an incubation time as short as 30 minutes could generate valid results at room temperature. Human serum control samples were run with an incubation time of 30 minutes and a development time of 30 minutes at either 1×, 2×, or 4× conjugate concentration and either 25° C. or 37° C. It was found that at 37° C., only a 2× concentration of conjugate was required to obtain target absorbance readings. However, target absorbance readings could also be obtained at 25° with a 4× conjugate concentration.

The fourth set of variables to be analyzed were conjugate concentration and development time. Human serum control samples were run at room temperature with a conjugate concentration of 1×, 2×, or 4× and a development time of 5, 10, 20, or 90 minutes. It was found that a development time as short as 20 minutes could result in valid readings at a 2× conjugate concentration, and that this time could be cut to 10 minutes with a 4× conjugate concentration.

To establish the minimal incubation time required for valid readings at room temperature, different combinations of incubation time, development time, and conjugate concentration were tested. It was found that optimal results could be achieved with an incubation time of 20 minutes, a development time of 20 minutes, and a conjugate concentration of 2×.

Based on the above results (discussed in greater detail in the Examples below), a method for rapid detection and measurement of sPLA₂ levels in a biological fluid sample was developed. This method may be used to measure sPLA₂ levels in any biological fluid. In certain preferred embodiments, the biological fluid is serum. The Cayman kit has not been validated for use with serum samples. As shown in the Examples section, the rapid measurement methods disclosed herein were found to be capable of measuring serum sPLA₂ levels as low as 3.1 ng/ml. Therefore, in certain embodiments, the rapid measurement methods disclosed herein may be utilized to measure sPLA₂ levels ranging from about 3 ng/ml to about 100 ng/ml.

In certain embodiments, the rapid sPLA₂ detection and measurement methods disclosed herein utilize incubation times of about 15 to about 60 minutes, substantially shorter than the overnight incubation period required by the Cayman kit. In certain preferred embodiments, the incubation time is about 15 to about 30 minutes and in more preferred embodiments about 15 to about 25 minutes. In certain embodiments, the methods disclosed herein have reduced development times as well, such as for example about 15 to about 25 minutes. In certain embodiments, the total running time of the assay, from test sample loading to measurement of absorbance values, is about 120 minutes or less. In certain of these embodiments, the total running time is about 100 minutes or less, and in certain embodiments the total running time is about 80 minutes or less. It was previously thought that ELISA-type assays were incapable of providing results in this short of a time period, and as such were impractical for clinical use (Styles 2000).

In certain embodiments, all steps of the rapid sPLA₂ detection and measurement method disclosed herein may be performed at room temperature. This provides an additional advantage over the Cayman kit because the Cayman kit requires incubation at 4° C., which can be impractical in a clinical or hospital setting.

In addition to the rapid detection and measurement methods disclosed herein, methods have been developed for detecting and measuring sPLA₂ levels in urine. These methods utilize a protocol that is similar to the rapid detection and measurement disclosed herein, but with less emphasis on the shortened incubation time. As shown in the Examples section below, standard curves generated using urine samples from three subjects were very similar to those obtained using sPLA₂-spiked buffer only, indicating that the urine protocol disclosed herein provides an accurate measure of sPLA₂ levels. The Cayman kit has not been validated for use with urine samples. Urine is a complex fluid that varies significantly from sample to sample. For example, urea, pH, salt content, osmolarity, and other factors may be substantially different between two test samples. Changes in these characteristics can lead to interference in the antibody/antigen interaction that is necessary for ELISA based assays. Therefore, the ability of the assay disclosed herein to accurately measure urine sPLA₂ levels is unexpected.

In certain embodiments of the sPLA₂ detection and measurement assay disclosed herein, one or more biological fluid test samples from a subject are applied to a plate coated with a capture antibody that specifically binds sPLA₂. The biological fluid may be selected from the group consisting of, but not limited to, serum, urine, plasma, or synovial fluid. Preferably, the biological fluid is serum or urine. The capture antibody on the plate may bind a specific sPLA₂ polypeptide, such as for example sPLA₂ type IIA. In certain embodiments, test samples may be diluted prior to loading onto the plate. For example, the samples may be diluted from about 1:10 to about 1:10,000 in a diluent. In certain of these embodiments, the samples are diluted 1:10 in diluent. The diluent may consist of buffer only. Alternatively, the diluent may comprise additional substituents, such as for example mouse or human serum. In certain embodiments, a single test sample may be loaded onto the plate multiple times, such as for example in duplicate or triplicate.

One or more quality control samples may be loaded onto the plate along with the test samples. Quality control samples may include one or more positive controls containing various concentrations of sPLA₂ and/or one or more negative controls containing buffer only. In those embodiments wherein the test samples are diluted in a diluent, one or more of the quality control samples may be diluted to the same degree in the same diluent. Quality control samples can be used to verify the accuracy of the assay system. For example, in embodiments wherein a kit is provided for performing the rapid detection and measurement methods disclosed herein, one or more quality control samples may be included with the kit so that the user can verify reagent quality and assay accuracy. In other embodiments, such a kit may include instructions for the user to generate their own set of quality control samples.

An acetylcholinesterase (AChE) conjugate is applied to each sample on the plate, wherein the conjugate specifically binds to sPLA₂ at an epitope distinct from that bound by the capture antibody. In certain embodiments, the conjugate comprises AChE conjugated to an antibody or antibody fragment, such as for example Fab′. In certain embodiments, the conjugate is loaded at a concentration from about 2× to about 4× the conjugate concentration utilized in the Cayman kit.

After addition of conjugate, samples are incubated at room temperature for about 15 to about 60 minutes for the rapid detection and measurement method. In certain preferred embodiments, the incubation time for the rapid detection and measurement assay is about 15 to about 30 minutes, and in more preferred embodiments it is about 15 to about 25 minutes. For the urine assay, samples may be incubated at room temperature for about 15 minutes to about 24 hours.

After incubation, wells are washed one or more times, and ElIman's reagent is added to each well for the development step. For the rapid detection and measurement assay, the plate is developed at room temperature for about 15 minutes to about 25 minutes. For the urine assay, samples may be developed at room temperature for about 30 minutes to about 24 hours. In certain embodiments of the urine assay, the samples may be developed for about 30 minutes to about 90 minutes. After development, the absorbance of each well is measured at about 400 to about 420 nm. In certain preferred embodiments, absorbance is measured at about 405 to about 412 nm.

In certain embodiments, the concentration of sPLA₂ in the test samples may be determined using a standard curve that plots absorbance versus sPLA₂ concentration. The standard curve is generated by subjecting one or more standard samples having various known sPLA₂ concentrations to the assay. In certain embodiments, the one or more standard samples may be loaded onto the same plate as the test samples, so that absorbance readings for the standard samples and test samples are obtained at or around the same time. In other embodiments, the standard samples may be run on a separate plate from the test samples. In certain of these embodiments, the standard samples may be run prior to the test samples, and the standard curve may be generated prior to running the test samples. For example, in embodiments wherein a kit is provided for performing the rapid detection and measurement methods disclosed herein, the standard curve may be provided with the kit. In other embodiments, the kit may contain standard samples that the user may utilize to generate a standard curve. Alternatively, the kit may contain a chart or diagram correlating absorbance reading to sPLA₂ concentration, so that the kit user does not have to employ the standard curve. In those embodiments wherein one or more quality control samples are run at the same time as the test samples, the assay is considered valid if the absorbance of the quality control samples is 80 to 120% that of the predicted absorbance for the quality control sample as determined by the standard curve.

In addition to or in lieu of determining the precise concentration of sPLA₂ in the test samples, the methods described above may be used to determine whether sPLA₂ is present in the test samples, and/or whether the concentration of sPLA₂ in the test samples is at or above a threshold concentration. To determine whether a test sample has an sPLA₂ concentration at or above a threshold level, the absorbance reading for each test sample is compared to the average absorbance reading for one or more standard samples with a specific threshold sPLA₂ concentration. In certain embodiments, the threshold sPLA₂ concentration may be 5 ng/ml, 10 ng/ml, 15 ng/ml, 25 ng/ml, 30 ng/ml, 40 ng/ml, 50 ng/ml, 60 ng/ml, 70 ng/ml, 80 ng/ml, 90 ng/ml, or 100 ng/ml. In certain embodiments, a test sample is considered to have an sPLA₂ concentration at or above the threshold concentration represented by a standard sample if it has an absorbance reading that is 80% or greater than the average absorbance reading for the standard sample. In certain of these embodiments, a test sample may be considered at or above the threshold concentration represented by the standard sample if it has an absorbance reading that is 90% or greater than that of the average absorbance reading for the standard sample, and in other embodiments if it has an absorbance reading that is 100% or greater than the average absorbance reading for the standard sample.

The methods disclosed herein may be used to measure the level of sPLA₂ generally, or to measure the level of one or more sPLA₂ subtypes. For example, the methods disclosed herein may be used to measure the concentration of sPLA₂ type IIA in a test sample by utilizing capture antibody and/or conjugate that specifically bind sPLA₂ type IIA. In those embodiments wherein the capture antibody targets a specific sPLA₂ subtype, the antibody preferably exhibits little or no cross-reactivity to other sPLA₂ polypeptides.

In certain embodiments, the methods provided herein may be used to diagnose a subject with a condition associated with elevated sPLA₂ levels. A “condition associated with elevated sPLA₂ levels” as used herein includes, for example, a condition associated with inflammation such as for example atherosclerosis, coronary artery disease, multiple sclerosis, Alzheimer's disease, sickle cell disease, rheumatoid arthritis, or osteoarthritis. In these embodiments, the methods may be utilized alone or in combination with other known assays or methods for diagnosing such conditions. In certain of these embodiments, an elevated sPLA₂ level refers to an sPLA₂ level of 10 ng/ml or greater in a biological fluid. In certain preferred embodiments, an elevated sPLA₂ level refers to an sPLA₂ level of 25 ng/ml, in other preferred embodiments 50 ng/ml or greater, in other preferred embodiments 75 ng/ml or greater, and in still other preferred embodiments 100 ng/ml or greater. In certain embodiments of the diagnosis methods disclosed herein, the modified ELISA immunoassay disclosed herein may be used to measure the precise concentration of sPLA₂ in a test sample. In other embodiments, the immunoassay may be used to determine whether the test sample has an sPLA₂ concentration that is at or above a concentration that is deemed to be elevated.

In certain embodiments, the methods provided herein may be used to monitor treatment of a subject diagnosed with a condition associated with elevated sPLA₂ levels. In these embodiments, the method may be employed at various time intervals after treatment to evaluate treatment progress.

In certain embodiments, the methods disclosed herein do not require the immobilization of capture antibodies on an ELISA plate. Rather, the capture antibodies may be immobilized on a strip or other matrix. For example, antibodies may be immobilized on a strip and then exposed to a biological fluid test sample, such as for example a urine test sample. Such embodiments are convenient for use in a home setting, or for rapid diagnosis in a clinical setting. In one embodiment, a strip may include a membrane pre-coated with capture antibodies on a test region of the strip. When a test sample is applied to the test region, sPLA₂ in the test sample binds to the capture antibody. Conjugate is then applied to the test region, where it binds to an epitope on sPLA₂ that is not bound by the capture antibody. In certain embodiments, the conjugate is already present on the strip prior to sample application. sPLA₂-bound conjugate is then used to detect sPLA₂ either directly or indirectly. For example, the conjugate may be an AChE conjugate. In these embodiments, the conjugate may be detected by applying ElIman's reagent and monitoring color development and/or intensity over time, either visually or using a detection device. Color development and/or intensity may be used to detect the presence or concentration of sPLA₂ in the sample, or to determine whether the sPLA₂ concentration in the sample is at or above a threshold concentration. In certain embodiments, the color of the strip may be compared to the color of one or more control strips that have been treated with samples of known sPLA₂ concentration. In other embodiments, different types of strips or matrixes may be implemented with the above methods. These strips may be used to detect the presence of sPLA₂ in a sample, determine whether a sample has an sPLA₂ concentration that is at or above a threshold level, or determine the sPLA₂ concentration or concentration range of a sample. For example, to determine whether. In other embodiments, the conjugate may contain colloidal gold particles pre-coated with capture antibody. In these embodiments, the test sample moves across the membrane chromatographically by capillary action, and migrates to a test region of the strip coated with capture antibody.

### EXAMPLE 1

### Effect on Serum Assay of Altering Incubation Time and Temperature

In the Cayman assay, incubation of sample with capture antibody and conjugate is performed overnight at 4° C. Incubation time and incubation temperature were modified in an attempt to develop an assay that could be performed in less time and at room temperature.

sPLA₂ standard samples (Cayman catalog no. 485004) were reconstituted in 10% normal human serum (Sigma-Aldrich catalog no. H4522) and EIA buffer (Cayman catalog no. 400060) at 0, 2.5, 5, and 10 ng/ml (equivalent to 0, 25, 50, and 100 ng/ml neat serum concentration, respectively). Cayman EIA buffer is 1 M phosphate, pH 7.0, 1% BSA, 4 M NaCl, 10 mM EDTA, and 0.1% sodium azide. Standard samples were loaded in duplicate onto a Cayman sPLA₂ (human type IIA) EIA plate (Cayman catalog no. 485002) at a volume of 100 μl/well. This plate is pre-coated with monoclonal sPLA₂ type IIA capture antibody. Conjugate was reconstituted in EIA buffer and added to each well at a volume of 100 μl/well, for a final concentration of 2×. The plate was incubated on a plate shaker for 30, 45, 60, or 90 minutes at 4° C., 25° C., or 37° C. in order to determine optimal incubation time and temperature. The wells were washed five to six times with wash buffer (Cayman catalog no. 400062), 200 μl reconstituted Ellman's reagent was added to each well, and plates were developed in the dark on a plate shaker for 20 minutes. After development, the absorbance of each well was measured at Abs₄₀₅ using a micro-well plate reader.

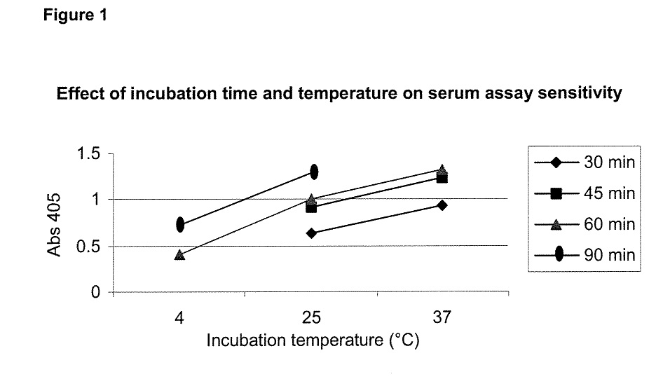

The results of this experiment are set forth in FIG. 1. The target absorbance value was between 0.5 and 1.6 AU at Abs₄₀₅. Increasing the incubation temperature up to 37° C. and increasing incubation time up to 90 minutes resulted in increased sPLA₂ binding as measured by increasing Abs₄₀₅. Nevertheless, target absorbance readings were observed at the shortest incubation time tested (30 minutes) at room temperature (25° C.). These results establish that the incubation step can be run at room temperature in substantially less time than that required by the standard Cayman kit.

### EXAMPLE 2

### Effect on Serum Assay of Altering Incubation Time, Development Time, and Conjugate Concentration

The ELISA assay was repeated essentially as described in Example 1, but with variations in incubation time, development time, and conjugate concentration in order to determine optimal assay conditions. Incubation times of 30, 45, 60, or 90 minutes, development (dvlpt.) times of 5, 10, 20, or 90 minutes, and conjugate concentrations of 2× or 4× were evaluated. The incubation temperature was 25° C. Target Abs₄₀₅ values were again 0.5-1.6 AU. The results of this experiment are summarized in Table 1. Samples falling within target absorbance values are in italics.

TABLE 1

| | 4X | | | | 2X | | | | |
|---|---|---|---|---|---|---|---|---|---|
| | 90 | 60 | 45 | 30 | 90 | 60 | 45 | 30 | |
| Incubation time | min | min | min | min | min | min | min | min | Dvlpt. |
| Incubation temp. | 25° C. | 25° C. | 25° C. | 25° C. | 25° C. | 25° C. | 25° C. | 25° C. | time |
| | 0.584 | 0.427 | 0.409 | 0.294 | 0.370 | 0.251 | 0.238 | 0.178 | 5 min |
| | 0.834 | 0.651 | 0.563 | 0.376 | 0.490 | 0.300 | 0.281 | 0.188 | 10 min |
| | 1.280 | 1.012 | 0.924 | 0.638 | 0.801 | 0.512 | 0.468 | 0.310 | 20 min |
| | 1.926 | 1.944 | 1.933 | 1.838 | 1.970 | 1.670 | 1.597 | 1.218 | 90 min |

As shown in Table 1, increasing incubation time and development time up to 90 minutes and conjugate concentration up 4× resulted in increased sPLA₂ binding as measured by increasing absorbance values (Abs₄₀₅). Nevertheless, target absorbance values were achieved at, for example, an incubation time of 30 minutes and a development time of 20 minutes at 4× conjugate concentration, and at an incubation time of 60 minutes and a development time of 20 minutes at 2× conjugate concentration.

### EXAMPLE 3

### Effect on Serum Assay of Altering Conjugate Concentration and Incubation Temperature

The ELISA assay was repeated essentially as described in Example 1, but with variations in conjugate concentration and incubation temperature in order to determine optimal assay conditions. Conjugate concentrations of 1×, 2×, or 4× and incubation temperatures of 25° C. or 37° C. were evaluated. The incubation time was 30 minutes and the development time was 20 minutes. Target Abs₄₀₅ values were again 0.5-1.6 AU.

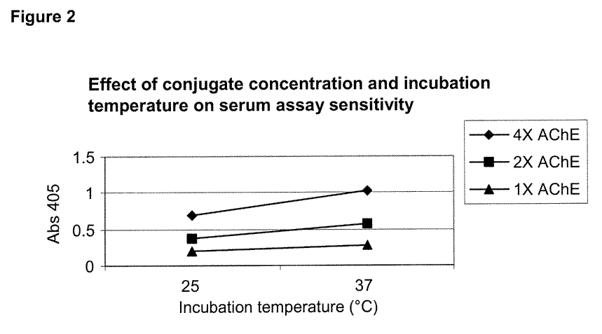

The results of this experiment are set forth in FIG. 2. As illustrated therein, the assay was effective with an incubation temperature of 25° C. (i.e., room temperature) when a higher conjugate concentration (4×) was used. At an incubation temperature of 37° C., a 2× conjugate concentration was sufficient to produce target absorbance values.

### EXAMPLE 4

### Effect on Serum Assay of Altering Conjugate Concentration and Development Time

The ELISA assay was repeated essentially as described in Example 1, but with variations in conjugate concentration and development time to determine the effects of these modifications on assay performance. Conjugate concentrations of 1×, 2×, or 4× and development times of 5, 10, 20, or 90 minutes were used. The incubation time was 30 minutes and the incubation temperature was 25° C. Target Abs₄₀₅ values were again 0.5-1.6 AU.

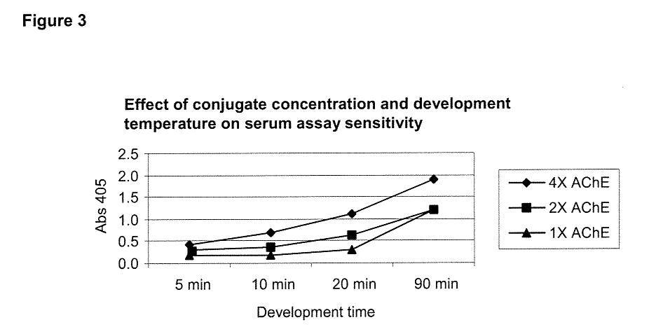

The results of this experiment are set forth in FIG. 3. As shown therein, target absorbance readings were obtained with a development time as short as 20 minutes with a 2× conjugate concentration or at 10 minutes with a 4× conjugate concentration.

### EXAMPLE 5

### Determination of Minimum Required Incubation and Development Times for Serum Assay

The ELISA assay was repeated essentially as described in Example 1, but with variations in incubation time, development time, and conjugate concentration to determine the minimum requirements for an effective assay. The times chosen included a window of +/−5 minutes, and target Abs₄₀₅ values were once again 0.5 and 1.6 AU. The results of this assay are summarized in Tables 2 and 3.

TABLE 2

| | Incubation | | Dvlpt. | | |
|---|---|---|---|---|---|
| [Conjugate] | time | Range | Time | Range | Total time |
| 4X | 15 min | 10-20 min | 15 min | 10-20 min | 30 min |
| 2X | 15 min | 10-20 min | 25 min | 20-30 min | 40 min |
| 2X | 20 min | 15-25 min | 20 min | 15-25 min | 40 min |
| 2X | 25 min | 20-30 min | 15 min | 10-20 min | 40 min |

TABLE 3

Sample incubation time

| 15 min | 20 min | 25 min | Dvlpt. time |
|---|---|---|---|
| 0.555 | 0.763 | 0.912 | 15 min |
| 0.540 | 0.735 | 1.054 | 15 min |
| 0.653 | 0.911 | 1.107 | 20 min |
| 0.652 | 0.871 | 1.315 | 20 min |
| 0.817 | 1.126 | 1.394 | 25 min |
| 0.771 | 1.078 | 1.603 | 25 min |

Since a development of less than 20 minutes decreased assay sensitivity, the following conditions were chosen for a follow-up validation assay: 20 minute incubation time, 20 minute development time, and conjugate concentration of 2×. Using these times, the total running time of the assay was expected to be approximately 80 minutes. This estimate takes into account 15 minutes for sample preparation, buffer preparation, and the like, 5 minutes for sample addition, 5 minutes for the wash step following incubation, 5 minutes to add substrate (i.e., Ellman's reagent), 5 minutes to obtain absorbance readings following development, and 5 minutes for data analysis.

### EXAMPLE 6

### Determination of Sensitivity and Range of Serum Assay

The sensitivity of the modified serum sPLA₂ assay was determined by running the ELISA assay set forth in Example 1 using the conditions developed in Examples 1-5. Specifically, this sensitivity experiment utilized an incubation time of 20 minutes+/−5 minutes, a development time of 20 minutes+/−5 minutes, and a conjugate concentration of 2×. The entire assay was performed at 25° C. The results of this experiment are summarized in Table 4. “Lowest positive reading” refers to the lowest concentration at which a positive reading was obtained. A positive reading was any absorbance reading at least 1.5 times greater than background.

TABLE 4

| Lowest positive reading | Serum equivalent | |
|---|---|---|
| (ng/ml) | (ng/ml) | Dvlpt. time |
| 0.63 | 6.3 | 10 min |
| 0.31 | 3.1 | 15 min |
| 0.31 | 3.1 | 20 min |
| <0.31 | <3.1 | 25 min |

As shown in Table 4, the lowest sample concentration at which a positive reading was obtained was 0.31 ng/ml. This is equivalent to a serum concentration of 3.1 ng/ml, because the test samples were diluted at 1/10.

### EXAMPLE 7

### Determination of Percent Recovery of sPLA₂ in 10% Normal Serum

The percent recovery of sPLA₂ standard in 10% normal human serum using two different sources of normal serum (Chemicon, Sigma-Aldrich) for 20 and 30 minute development times was determined. Incubation times were 20 to 30 minutes, and both incubation and development took place at room temperature. Recovery values ranged from 88-95%, with similar recovery in both serum samples. These results are acceptable for this type of assay.

### EXAMPLE 8

### Modified Assay for Detecting sPLA₂ in a Serum Sample

Based on the information developed in Examples 1-7, the following modified assay protocol for detecting and measuring sPLA₂ in serum was developed.

EIA buffer is generated by diluting 10 ml of 10×EIA Buffer Concentrate from the Cayman kit into 90 ml ACS grade water. Wash buffer is made by diluting 5 ml of Wash Buffer Concentrate from the Cayman kit into ACS grade water to a final volume of 2 L, then adding 5 ml of 20% Tween 20. Conjugate (100 dtn) is reconstituted in 5 ml of EIA buffer and stored at 2-8° C. until use. Ellman's Reagent (100 dtn) is reconstituted in 20 ml ACS grade water.

sPLA₂ (human) EIA Standard (positive control) at concentrations of 10 ng/ml, 5 ng/ml, and 2.5 ng/ml in 10% Normal Human Serum (VWR, Cat. No. 14230-706) is thawed at room temperature (approximately 15-30° C.) for no longer than two hours and stored at 2-8° C. until use. A negative control standard of 10% Normal Human Serum in EIA buffer is thawed and stored in the same manner. After thawing, the bottom of each vial is tapped on a hard surface to ensure that the contents moved to the bottom of the vial.

An EIA Plate is brought to room temperature for approximately ten minutes. This plate is pre-coated with capture antibody specific for an sPLA₂ protein. All standard wells and test wells are loaded in duplicate or triplicate. Standard samples are added to the wells as follows: wells 1A and 2A, 100 μl/well 10% Normal Human Serum (negative control standard; wells 1B and 2B, 100 μl/well 2.5 ng/mL sPLA₂ (human) EIA Standard; wells 1C and 2C, 100 μl/well 5 ng/mL sPLA₂ (human) EIA Standard; wells 1D and 2D, 100 μl/well 10 ng/mL sPLA₂ (human) EIA Standard.

AChE Conjugate is added to all wells at 100 μl/well and at a concentration of 2× or 4×, and the plate is covered and incubated on a plate shaker at 15-30° C. for 15-60 minutes, and preferably 15-25 minutes. Wells are then emptied and washed five times using wash buffer. After the final wash, excess liquid is removed from the plate using paper towels, and 200 μl of Ellman's Reagent is added to each well. The plate is covered with aluminum foil and developed on a plate shaker at room temperature (15-30° C.) for 15-25 minutes. The plate is then transferred to a plate reader set at an absorbance value of 405 nm, and absorbance readings are obtained for each well.

In order for a plate reading to be considered valid, the following criteria must be met: 1) absorbance readings for duplicate samples must be within 25% of one another; 2) 10 ng/ml sPLA₂ (human) EIA Standard must have an average absorbance value greater than 0.5 but less than 2.0 AU; 5 ng/ml sPLA₂ (human) EIA Standard must have an average absorbance value greater than the negative control and 40-80% of the 10 ng/ml sPLA₂ (human) EIA Standard.

A standard curve is generated using valid readings from the standard samples, with absorbance on the Y-axis and standard concentration on the X-axis. The equation of the resultant line is used to determine the concentration of the test samples using the following formula:

sPLA₂(Type IIa)EIA Concentration(pg/ml)=[(Abs₄₀₅−Y−intercept)/slope]×dilution.

### EXAMPLE 9

### Confirmation of Serum Assay Efficacy in Clinical Setting

The serum assay developed in Example 8 was tested in a clinical setting using test samples from three different subjects to determine whether sPLA₂ levels in the test samples were at or above a threshold concentration of 100 ng/ml.

Incubation and development were carried out at 24° C. for the first subject, room temperature for the second subject, and 21° C. for the third subject. Incubation times were 60 minutes for the first subject, 50 minutes for the second subject, and 20 minutes for the third subject. The development time was 20 minutes for each subject.

For each subject, test sample and standard samples were loaded in triplicate onto a plate. Four control samples were utilized for each plate: a negative control (0 ng/ml) and three positive controls (25 ng/ml, 50 ng/ml, and 100 ng/ml). The results for each plate are set forth in Tables 5-7.

TABLE 5
Subject 1

| | |
|---|---|
| Control | Abs₄₀₅ |

| sample | | Sample 1 | Sample 2 | Sample 3 | Mean |
|---|---|---|---|---|---|
| 0 | ng/ml | 0.175 | 0.164 | 0.161 | 0.167 |
| 25 | ng/ml | 0.406 | 0.365 | 0.383 | 0.385 |
| 50 | ng/ml | 0.545 | 0.525 | 0.495 | 0.522 |
| 100 | ng/ml | 0.799 | 0.710 | 0.829 | 0.779 |

| | | | | |
|---|---|---|---|---|
| Test sample | 1.012 | 0.921 | 0.897 | 0.943 |

TABLE 6
Subject 2

| | |
|---|---|
| Control | Abs₄₀₅ |

| sample | | Sample 1 | Sample 2 | Sample 3 | Mean |
|---|---|---|---|---|---|
| 0 | ng/ml | 0.244 | 0.172 | 0.180 | 0.198 |
| 25 | ng/ml | 0.387 | 0.382 | 0.399 | 0.389 |
| 50 | ng/ml | 0.573 | 0.546 | 0.517 | 0.545 |
| 100 | ng/ml | 0.816 | 0.819 | 0.812 | 0.816 |

| | | | | |
|---|---|---|---|---|
| Test sample | 1.303 | 1.303 | 1.353 | 1.320 |

TABLE 7
Subject 3

| | |
|---|---|
| Control | Abs₄₀₅ |

| sample | | Sample 1 | Sample 2 | Sample 3 | Mean |
|---|---|---|---|---|---|
| 0 | ng/ml | 0.124 | 0.163 | 0.162 | 0.150 |
| 25 | ng/ml | 0.544 | 0.577 | 0.599 | 0.573 |
| 50 | ng/ml | 0.871 | 0.881 | 0.943 | 0.898 |
| 100 | ng/ml | 1.393 | 1.338 | 1.416 | 1.382 |

| | | | | |
|---|---|---|---|---|
| Test sample | 1.784 | 1.829 | 1.812 | 1.808 |

In order for a particular assay to be considered valid, the following criteria had to be met: 1) mean absorbance reading of 0.5 to 2.0 for the 100 ng/ml control sample; 2) mean absorbance reading of less than 0.3 for the negative control sample; 3) mean absorbance reading for 50 ng/ml control sample greater than mean absorbance reading of negative control sample; and 4) mean absorbance reading for 50 ng/ml divided by mean absorbance reading for 100 ng/ml equals 0.40 to 0.80. Since all of these criteria were met for all three assays, all assays were considered valid. For a test sample to be classified as having an sPLA₂ concentration at or above the threshold level of 100 ng/ml, the mean absorbance reading of the test sample had to be greater than 90% of the mean absorbance reading of the 100 ng/ml control sample. All three samples met this criteria, meaning that all three were at or above the threshold concentration. A similar approach may be utilized to determine whether a test sample has an sPLA₂ concentration at or above a 50 or 25 ng/ml threshold (i.e., determine whether mean absorbance reading of the test sample was greater than 90% of the mean absorbance reading of the 50 or 25 ng/ml control sample, respectively.

### EXAMPLE 10

### Development of Assay for Measuring sPLA₂ Levels in Urine

20 ng sPLA₂ was reconstituted in 50 μl EIA buffer to generate a 400 ng/ml stock. This stock was used to generate four sets of standards. One set of standards was diluted in EIA buffer, while the other three were diluted in neat urine from one of three human subjects. Each set of standards contained eight samples: 6.00 ng/ml, 4.00 ng/ml, 2.00 ng/ml, 1.00 ng/ml, 0.50 ng/ml, 0.17 ng/ml, 0.06 ng/ml, and 0 ng/ml. The 0 ng/ml sample (“standard H”) contained diluent only. The dilution strategy for the other standard samples was as follows:
5.25 μl 400 ng/ml stock into 344.75 μl diluent to generate 6.00 ng/ml standard A;
100 μl 6.00 ng/ml standard A into 50 μl diluent to generate 4.00 ng/ml standard B;
75 μl 4.00 ng/ml standard B into 75 μl diluent to generate 2.00 ng/ml standard C;
75 μl 2.00 ng/ml standard C into 75 μl diluent to generate 1.00 ng/ml standard D;
75 μl 1.00 ng/ml standard D into 75 μl diluent to generate 0.50 ng/ml standard E;
50 μl of 0.50 ng/ml standard E into 100 μl diluent to generate 0.17 ng/ml standard F; and
54 μl 0.17 ng/ml standard F into 96 μl diluent to generate 0.06 ng/ml standard G.
   Each sample was loaded in duplicate onto the 96-well EIA plate from the Cayman kit at a volume of 30 μl/well.

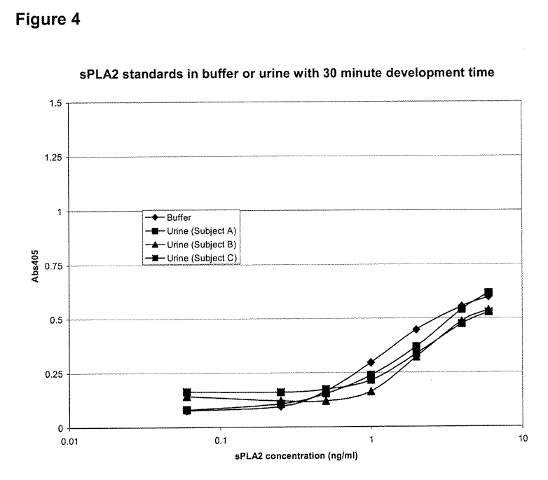

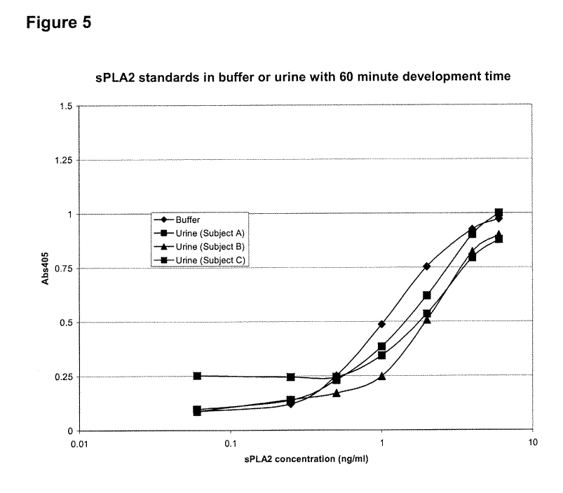

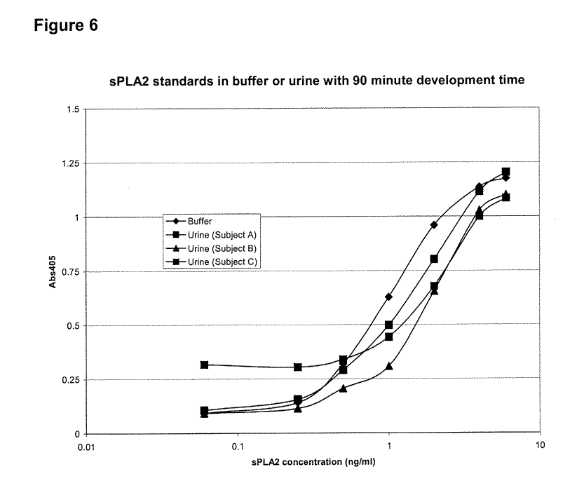

AChE-Fab′ conjugate was reconstituted in 20 ml of EIA buffer to a final concentration one half that recommended by the Cayman kit, and 30 μl reconstituted conjugate was added to each well. The plate was incubated on a plate shaker overnight 2-8° C. The wells were washed with wash buffer, reconstituted Ellman's reagent was added to each well, and plates were developed in the dark on a plate shaker for 30, 60, or 90 minutes. After development, the absorbance of each well was measured at Abs₄₀₅ using a micro-well plate reader. The results of this assay are summarized in Tables 8-10. Standard curves were generated for each set of standards at each development time (FIGS. 4-6). Mean absorbance was plotted on the Y-axis, and sPLA₂ concentration was plotted on the X-axis. The standard curves generated for each of the three test subjects were very similar to those obtained with buffer only.

### EXAMPLE 11

### Determination of Reagent Stability

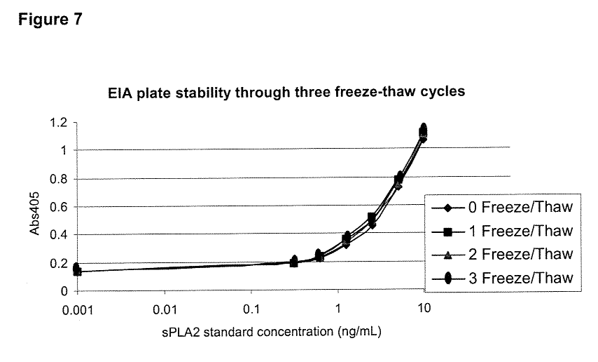

The stability of various components used in the ELISA assays described herein was evaluated. Specifically, the effect of zero to three freeze/thaw cycles on sPLA₂ standard, conjugate, Ellman's reagent, and EIA plate stability was evaluated using 10, 20, and 30 minute development times. These results are summarized in Tables 11-13 and FIG. 7.

TABLE 11

| Standard | | 10 min avg. | 20 min avg. | 30 min avg. |
|---|---|---|---|---|
| 0 | freeze/thaw | 0.665 | 1.143 | 1.726 |
| 1 | freeze/thaw | 0.666 | 1.153 | 1.657 |
| 2 | freeze/thaw | 0.617 | 1.070 | 1.539 |
| 3 | freeze thaw | 0.652 | 1.112 | 1.598 |

| | | | |
|---|---|---|---|
| Overnight at 25° C. | 0.655 | 1.061 | 1.650 |

TABLE 12

| Conjugate | | 10 min avg. | 20 min avg. | 30 min avg. |
|---|---|---|---|---|
| 0 | freeze/thaw | 0.658 | 1.140 | 1.661 |
| 1 | freeze/thaw | 0.554 | 0.960 | 1.414 |
| 2 | freeze/thaw | 0.585 | 1.048 | 1.466 |
| 3 | freeze thaw | 0.610 | 1.151 | 1.602 |

| | | | |
|---|---|---|---|
| Overnight at 25° C. | 0.604 | 1.133 | 1.616 |

TABLE 13

| Ellman's reagent | | 10 min avg. | 20 min avg. | 30 min avg. |
|---|---|---|---|---|
| Control | | 0.527 | 1.001 | 1.464 |
| 4° C. | overnight in dark | 0.629 | 1.078 | 1.507 |
| 4° C. | overnight in light | 0.567 | 1.048 | 1.454 |
| 25° C. | overnight in dark | 0.646 | 1.048 | 1.513 |
| 25° C. | overnight in light | 0.633 | 1.056 | 1.458 |

As shown in Table 11, no significant difference in recovery of standards was observed following three freeze/thaw cycles or overnight storage at 250C. As shown in Table 12, no significant difference in recovery of conjugate was observed following three freeze/thaw cycles or overnight storage at 25° C. A decrease was observed for one or two freeze/thaw cycles, but since there was not a decrease for three freeze/thaw cycles, these decreases at one and two cycles were not interpreted as not significant. The conjugate will be supplied as powder reconstituted on site, so the freeze/thaw stability of the reconstituted solution will not be an issue. As shown in Table 13, no significant difference in recovery of ElIman's Reagent was observed following overnight storage at 4° C. or 25° C. in the light or dark. Overnight storage in light led to a slight reduction in absorbance values comparatively. As shown in FIG. 7, no significant difference in the effectiveness of the precoated EIA plate was observed after three freeze/thaw cycles.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. Therefore, it must be expressly understood that the illustrated embodiments have been shown only for the purposes of example and should not be taken as limiting the invention, which is defined by the following claims. All references cited herein are incorporated by reference as if fully set forth herein.

### REFERENCES

1. Boekholdt, S. M. et al. 2005. Arterioscler Thromb Vasc Biol 25:839.
2. Cunningham, T. J., et al. 2006. J. Neuroinflamm. Published online Sep. 11, 2006.
3. Hurt-Camejo, E., et al. 2001. Circulation Res 89:298.
4. Jamal, O, S., et al. 1998. Ann Rheum Dis 57:550-558)
5. Moses, G. S. D., et al. 2006. J Neuroinflamm 3:28.

TABLE 8
Absorbance at 405 nm for buffer and spiked urine samples with 30 minute development time

| | Buffer only | | | Urine (Subject A) | | | Urine (Subject B) | | | Urine (Subject C) | | |
|---|---|---|---|---|---|---|---|---|---|---|---|---|
| | (Abs₄₀₅) | | | (Abs₄₀₅) | | | (Abs₄₀₅) | | | (Abs₄₀₅) | | |
| | | | Mean | | | Mean | | | Mean | | | Mean |
| [sPLA₂] | 1 | 2 | (SD) | 1 | 2 | (SD) | 1 | 2 | (SD) | 1 | 2 | (SD) |
| 6.00 | 0.608 | 0.590 | 0.599 | 0.620 | 0.615 | 0.617 | 0.558 | 0.520 | 0.539 | 0.521 | 0.535 | 0.528 |
| ng/ml | | | (0.013) | | | (0.004) | | | (0.026) | | | (0.010) |
| 4.00 | 0.554 | 0.556 | 0.555 | 0.537 | 0.544 | 0.541 | 0.491 | 0.482 | 0.487 | 0.503 | 0.445 | 0.474 |
| ng/ml | | | (0.002) | | | (0.005) | | | (0.007) | | | (0.041) |
| 2.00 | 0.452 | 0.443 | 0.447 | 0.375 | 0.363 | 0.369 | 0.347 | 0.297 | 0.322 | 0.325 | 0.346 | 0.336 |
| ng/ml | | | (0.006) | | | (0.008) | | | (0.035) | | | (0.015) |
| 1.00 | 0.291 | 0.300 | 0.296 | 0.239 | 0.236 | 0.238 | 0.153 | 0.174 | 0.163 | 0.226 | 0.204 | 0.215 |
| ng/ml | | | (0.006) | | | (0.002) | | | (0.015) | | | (0.016) |
| 0.50 | 0.162 | 0.167 | 0.165 | 0.153 | 0.155 | 0.154 | 0.120 | 0.120 | 0.120 | 0.181 | 0.167 | 0.174 |
| ng/ml | | | (0.004) | | | (0.002) | | | (0.000) | | | (0.010) |
| 0.25 | 0.097 | 0.093 | 0.095 | 0.098 | 0.115 | 0.107 | 0.159 | 0.083 | 0.121 | 0.165 | 0.157 | 0.161 |
| ng/ml | | | (0.002) | | | (0.012) | | | (0.054) | | | (0.006) |
| 0.06 | 0.079 | 0.074 | 0.076 | 0.080 | 0.079 | 0.080 | 0.075 | 0.210 | 0.143 | 0.168 | 0.161 | 0.164 |
| ng/ml | | | (0.003) | | | (0.001) | | | (0.096) | | | (0.005) |
| 0 | 0.065 | 0.065 | 0.065 | 0.066 | 0.086 | 0.076 | 0.076 | 0.066 | 0.071 | 0.167 | 0.157 | 0.162 |
| ng/ml | | | (0.000) | | | (0.014) | | | (0.008) | | | (0.007) |

TABLE 9
Absorbance at 405 nm for buffer and spiked urine samples with 60 minute development time

| | Buffer only | | | Urine (Subject A) | | | Urine (Subject B) | | | Urine (Subject C) | | |
|---|---|---|---|---|---|---|---|---|---|---|---|---|
| | (Abs₄₀₅) | | | (Abs₄₀₅) | | | (Abs₄₀₅) | | | (Abs₄₀₅) | | |
| | | | Mean | | | Mean | | | Mean | | | Mean |
| [sPLA₂] | 1 | 2 | (SD) | 1 | 2 | (SD) | 1 | 2 | (SD) | 1 | 2 | (SD) |
| 6.00 | 0.981 | 0.966 | 0.973 | 0.999 | 1.001 | 1.000 | 0.925 | 0.875 | 0.900 | 0.871 | 0.885 | 0.878 |
| ng/ml | | | (0.010) | | | (0.002) | | | (0.035) | | | (0.010) |
| 4.00 | 0.926 | 0.925 | 0.925 | 0.894 | 0.907 | 0.901 | 0.825 | 0.816 | 0.821 | 0.842 | 0.747 | 0.794 |
| ng/ml | | | (0.000) | | | (0.009) | | | (0.007) | | | (0.067) |
| 2.00 | 0.762 | 0.744 | 0.753 | 0.629 | 0.610 | 0.619 | 0.516 | 0.498 | 0.507 | 0.546 | 0.526 | 0.536 |
| ng/ml | | | (0.013) | | | (0.013) | | | (0.012) | | | (0.014) |
| 1.00 | 0.482 | 0.493 | 0.487 | 0.386 | 0.383 | 0.385 | 0.231 | 0.265 | 0.248 | 0.367 | 0.322 | 0.344 |
| ng/ml | | | (0.008) | | | (0.002) | | | (0.024) | | | (0.032) |
| 0.50 | 0.246 | 0.256 | 0.251 | 0.229 | 0.233 | 0.231 | 0.171 | 0.171 | 0.171 | 0.285 | 0.254 | 0.245 |
| ng/ml | | | (0.007) | | | (0.003) | | | (0.000) | | | (0.013) |
| 0.25 | 0.123 | 0.119 | 0.121 | 0.129 | 0.148 | 0.139 | 0.181 | 0.101 | 0.141 | 0.254 | 0.236 | 0.245 |
| ng/ml | | | (0.003) | | | (0.013) | | | (0.056) | | | (0.013) |
| 0.06 | 0.089 | 0.085 | 0.087 | 0.096 | 0.095 | 0.096 | 0.086 | 0.087 | 0.087 | 0.259 | 0.245 | 0.252 |
| ng/ml | | | (0.003) | | | (0.001) | | | (0.001) | | | (0.010) |
| 0 | 0.067 | 0.067 | 0.067 | 0.069 | 0.106 | 0.088 | 0.088 | 0.070 | 0.079 | 0.257 | 0.236 | 0.246 |
| ng/ml | | | (0.000) | | | (0.026) | | | (0.013) | | | (0.014) |

TABLE 10
Absorbance at 405 nm for buffer and spiked urine samples with 90 minute development time

| | Buffer only | | | Urine (Subject A) | | | Urine (Subject B) | | | Urine (Subject C) | | |
|---|---|---|---|---|---|---|---|---|---|---|---|---|
| | (Abs₄₀₅) | | | (Abs₄₀₅) | | | (Abs₄₀₅) | | | (Abs₄₀₅) | | |
| | | | Mean | | | Mean | | | Mean | | | Mean |
| [sPLA₂] | 1 | 2 | (SD) | 1 | 2 | (SD) | 1 | 2 | (SD) | 1 | 2 | (SD) |
| 6.00 | 1.182 | 1.168 | 1.175 | 1.200 | 1.206 | 1.203 | 1.119 | 1.081 | 1.100 | 1.076 | 1.092 | 1.084 |
| ng/ml | | | (0.010) | | | (0.005) | | | (0.027) | | | (0.011) |
| 4.00 | 1.133 | 1.139 | 1.136 | 1.107 | 1.122 | 1.114 | 1.030 | 1.026 | 1.028 | 1.048 | 0.952 | 1.000 |
| ng/ml | | | (0.004) | | | (0.011) | | | (0.003) | | | (0.068) |
| 2.00 | 0.965 | 0.952 | 0.959 | 0.812 | 0.791 | 0.802 | 0.664 | 0.646 | 0.655 | 0.704 | 0.652 | 0.678 |
| ng/ml | | | (0.009) | | | (0.015) | | | (0.012) | | | (0.037) |
| 1.00 | 0.622 | 0.635 | 0.628 | 0.499 | 0.496 | 0.498 | 0.286 | 0.330 | 0.308 | 0.472 | 0.413 | 0.443 |
| ng/ml | | | (0.009) | | | (0.003) | | | (0.031) | | | (0.042) |
| 0.50 | 0.311 | 0.325 | 0.318 | 0.289 | 0.292 | 0.290 | 0.206 | 0.206 | 0.206 | 0.359 | 0.320 | 0.340 |
| ng/ml | | | (0.010) | | | (0.002) | | | (0.000) | | | (0.028) |
| 0.25 | 0.142 | 0.139 | 0.141 | 0.152 | 0.159 | 0.156 | 0.116 | 0.112 | 0.114 | 0.317 | 0.294 | 0.305 |
| ng/ml | | | (0.002) | | | (0.005) | | | (0.003) | | | (0.016) |
| 0.06 | 0.095 | 0.092 | 0.093 | 0.106 | 0.105 | 0.106 | 0.092 | 0.093 | 0.092 | 0.325 | 0.308 | 0.317 |
| ng/ml | | | (0.002) | | | (0.001) | | | (0.001) | | | (0.012) |
| 0 | 0.067 | 0.067 | 0.067 | 0.070 | 0.072 | 0.071 | 0.071 | 0.068 | 0.070 | 0.323 | 0.295 | 0.309 |
| ng/ml | | | (0.000) | | | (0.001) | | | (0.002) | | | (0.020) |

What is claimed is:

1. A method for rapid measurement of secretory phospholipase A₂ (sPLA₂) levels in a biological fluid test sample from a subject comprising:
a) obtaining one or more biological fluid test samples from a subject;
b) applying said test samples to one or more test wells on a plate, wherein said test wells are pre-coated with a capture antibody that specifically binds sPLA₂;
c) applying an acetylcholinesterase (AChE) conjugate antibody to said test wells, wherein said AChE conjugate antibody comprises AChE conjugated to an antibody that specifically binds sPLA₂ at a different epitope than the capture antibody;
d) covering and incubating said plate at 15 to 30° C. for 15 to 60 minutes;
e) pouring out the contents of said test wells and washing said test wells with a buffer;
f) adding ElIman's reagent to said test wells and developing said plate at 15 to 30° C. for 15 to 25 minutes;
g) measuring the absorbance of said test sample at 400 to 412 nm; and
h) determining the concentration of sPLA₂ in said test sample using a standard curve that plots absorbance versus sPLA₂ concentration for one or more standard samples of known sPLA₂ concentration.

2. The method of claim 1, wherein the absorbance of said standard samples for use in generating said standard curve is obtained from standard samples loaded onto the same plate as said test samples.

3. The method of claim 1, wherein said standard samples for use in generating said standard curve are not loaded onto the same plate as said test samples.

4. The method of claim 1, further comprising:
repeating steps b-g using one or more quality control samples containing a known control concentration of sPLA₂; and
comparing the absorbance of said one or more quality control samples to the expected absorbance for a sample of said known control concentration of sPLA₂ as determined using said standard curve;
wherein the results of said method are validated if the absorbance of said one or more quality control samples is 80% to 120% of their expected absorbance as determined using the standard curve.

5. The method of claim 4, wherein said quality control samples are loaded onto the same plate as said test samples.

6. The method of claim 1, wherein said biological fluid is selected from the group consisting of serum and urine.

7. The method of claim 1, wherein said incubation time in step (d) is 15 to 25 minutes.

8. The method of claim 1, wherein said test samples are diluted in buffer prior to application to said test wells.

9. The method of claim 1, wherein said method is used to diagnose a condition associated with elevated sPLA₂ levels.

10. A kit for performing the method of claim 1.

11. The kit of claim 10, wherein said kit comprises a standard curve.

12. A method for measuring secretory phospholipase A₂ (sPLA₂) levels in a urine test sample from a subject comprising:
a) obtaining one or more urine test samples from a subject;
b) applying said test samples to one or more test wells on a plate, wherein said test wells are precoated with a capture antibody that specifically binds sPLA₂;
c) applying an acetylcholinesterase (AChE) conjugate antibody to said test wells, wherein said AChE conjugate antibody comprises AChE conjugated to an antibody that specifically binds sPLA₂ at a different epitope than the capture antibody;
d) covering and incubating said plate at 15 to 30° C. for 15 minutes to 24 hours;
e) pouring out the contents of said test wells and washing said test wells with a buffer;
f) adding ElIman's reagent to said test wells and developing said plate at 15 to 30° C. for 30 minutes to 24 hours;
g) measuring the absorbance of said test sample at 400 to 412 nm; and
h) determining the concentration of sPLA₂ in said test sample using a standard curve that plots absorbance versus sPLA₂ concentration for one or more standard samples of known sPLA₂ concentration.

13. The method of claim 12, wherein the absorbance of said standard samples for use in generating said standard curve is obtained from standard samples loaded onto the same plate as said test samples.

14. The method of claim 12, wherein the standard samples for use in generating said standard curve are not loaded onto the same plate as said test samples.

15. The method of claim 12, further comprising:
repeating steps b-g using one or more quality control samples containing a known control concentration of sPLA₂; and
comparing the absorbance of said one or more quality control samples to the expected absorbance for a sample of said known control concentration of sPLA₂ as determined using said standard curve;
wherein the results of said method are validated if the absorbance of said one or more quality control samples is 80% to 120% of their expected absorbance as determined using the standard curve.

16. The method of claim 15, wherein said quality control samples are loaded onto the same plate as said test samples.

17. The method of claim 12, wherein said development time in step (f) is 30 minutes to 90 minutes.

18. The method of claim 12, wherein said test samples are diluted in buffer prior to application to said test wells.

19. The method of claim 12, wherein said method is used to diagnose a condition associated with elevated sPLA₂ levels.

20. A kit for performing the method of claim 12.

21. The kit of claim 20, wherein said kit comprises a standard curve.

22. A method for determining whether the level of secretory phospholipase A₂ (sPLA₂) in a biological fluid test sample from a subject is at or above a threshold level comprising:
a) obtaining one or more biological fluid test samples from a subject;
b) applying said test samples to one or more test wells on a plate, wherein said test wells are pre-coated with a capture antibody that specifically binds sPLA₂;
c) applying an acetylcholinesterase (AChE) conjugate antibody to said test wells, wherein said AChE conjugate antibody comprises AChE conjugated to an antibody that specifically binds sPLA₂ at a different epitope than the capture antibody;
d) covering and incubating said plate at 15 to 30° C. for 15 to 60 minutes;
e) pouring out the contents of said test wells and washing said test wells with a buffer;
f) adding ElIman's reagent to said test wells and developing said plate at 15 to 30° C. for 15 to 25 minutes;
g) measuring the test absorbance of said test sample at 400 to 412 nm; and
h) comparing said test absorbance to the average standard absorbance at 400 to 412 nm of one or more standard samples having a threshold sPLA₂ concentration;
wherein said test samples are classified as having an sPLA₂ concentration at or above said threshold sPLA₂ concentration if said test absorbance is at least 80% of said average standard absorbance.

23. The method of claim 22, wherein said biological fluid is selected from the group consisting of serum and urine.

24. The method of claim 22, wherein said average standard absorbance is obtained from standard samples loaded onto the same plate as said test samples.

25. The method of claim 22, wherein said average standard absorbance is obtained from standard samples that are not loaded onto the same plate as said test samples.

26. The method of claim 22, wherein said incubation time in step (d) is 15 to 25 minutes.

27. The method of claim 22, wherein said test samples are classified as having an sPLA₂ concentration at or above said threshold sPLA₂ concentration if said test absorbance is at least 90% of said average standard absorbance.

28. The method of claim 27, wherein said test samples are classified as having an sPLA₂ concentration at or above said threshold sPLA₂ concentration if said test absorbance is at least 100% of said average standard absorbance.

29. The method of claim 22, wherein said test samples are diluted in buffer prior to application to said test wells.

30. The method of claim 22, wherein said method is used to diagnose a condition associated with elevated sPLA₂ levels.

31. A kit for performing the method of claim 22.

32. A method for determining whether the level of secretory phospholipase A₂ (sPLA₂) in a urine test sample from a subject is at or above a threshold level comprising:
a) obtaining one or more urine test samples from a subject;
b) applying said test samples to one or more test wells on a plate, wherein said test wells are pre-coated with a capture antibody that specifically binds sPLA₂;
c) applying an acetylcholinesterase (AChE) conjugate antibody to said test wells, wherein said AChE conjugate antibody comprises AChE conjugated to an antibody that specifically binds sPLA₂ at a different epitope than the capture antibody;
d) covering and incubating said plate at 15 to 30° C. for 15 minutes to 24 hours;
e) pouring out the contents of said test wells and washing said test wells with a buffer;
f) adding Ellman's reagent to said test wells and developing said plate at 15 to 30° C. for 30 minutes to 24 hours;
g) measuring the test absorbance of said test sample at 400 to 412 nm; and
h) comparing said test absorbance to the average standard absorbance at 400 to 412 nm of one or more standard samples having a threshold sPLA₂ concentration;
wherein said test samples are classified as having an sPLA₂ concentration at or above said threshold sPLA₂ concentration if said test absorbance is at least 80% of said average standard absorbance.

33. The method of claim 32, wherein said average standard absorbance is obtained from standard samples loaded onto the same plate as said test samples.

34. The method of claim 32, wherein said average standard absorbance is obtained from standard samples that are not loaded onto the same plate as said test samples.

35. The method of claim 32, wherein said development time in step (f) is 30 minutes to 90 minutes.

36. The method of claim 32, wherein said test samples are classified as having an sPLA₂ concentration at or above said threshold sPLA₂ concentration if said test absorbance is at least 90% of said average standard absorbance.

37. The method of claim 36, wherein said test samples are classified as having an sPLA₂ concentration at or above said threshold sPLA₂ concentration if said test absorbance is at least 100% of said average standard absorbance.

38. The method of claim 32, wherein said test samples are diluted in buffer prior to application to said test wells.

39. The method of claim 32, wherein said method is used to diagnose a condition associated with elevated sPLA₂ levels.

40. A kit for performing the method of claim 32.

41. A method for measuring secretory phospholipase A₂ (sPLA₂) levels in a urine test sample from a subject comprising:
a) obtaining a urine test sample from a subject;
b) applying said test sample to a strip, wherein the surface of said strip comprises a capture antibody that specifically binds sPLA₂;
c) applying an acetylcholinesterase (AChE) conjugate antibody to said strip, wherein said AChE conjugate antibody comprises AChE conjugated to an antibody that specifically binds sPLA₂ at a different epitope than the capture antibody;
d) incubating said strip at 15 to 30° C. for 15 minutes to 24 hours;
e) washing said strip with a buffer;
f) applying Ellman's reagent to said strip and developing said strip at 15 to 30° C. for 15 minutes to 24 hours;
g) observing the color of said strip following development;
h) determining the concentration of sPLA₂ in said test sample by comparing the color of said strip following incubation to the color of one or more standards.

42. The method of claim 41, wherein said incubation time in step (d) is 15 to 60 minutes.

43. The method of claim 42, wherein said incubation time in step (d) is 15 to 25 minutes.

44. The method of claim 41, wherein said development time in step (f) is 15 minutes to 90 minutes.

45. The method of claim 44, wherein said development time in step (f) is 30 minutes to 90 minutes.

46. The method of claim 41, wherein said biological fluid is selected from the group consisting of serum and urine.

47. The method of claim 41, wherein said test sample is considered to have an sPLA₂ concentration at or above a threshold level if the color of said color of said strip following development is:
a) the same as the color of a strip that has been treated with a standard sample having a known sPLA₂ concentration that is equal to a threshold level; or
b) the same as the color of a strip that has been treated with a standard sample having a known sPLA₂ concentration that is greater than a threshold level.

48. A method for diagnosing a subject with a condition associated with elevated secretory phospholipase A₂ (sPLA₂) levels comprising:
a) obtaining one or more biological fluid test samples from a subject;
b) applying said test samples to one or more test wells on a plate, wherein said test wells are pre-coated with a capture antibody that specifically binds sPLA₂;
c) applying an acetylcholinesterase (AChE) conjugate antibody to said test wells, wherein said AChE conjugate antibody comprises AChE conjugated to an antibody that specifically binds sPLA₂ at a different epitope than the capture antibody;
d) covering and incubating said plate at 15 to 30° C. for 15 minutes to 24 hours;
e) pouring out the contents of said test wells and washing said test wells with a buffer;
f) adding ElIman's reagent to said test wells and developing said plate at 15 to 30° C. for 15 minutes to 24 hours;
g) measuring the test absorbance of said test sample at 400 to 412 nm; and
h) comparing said test absorbance to the average standard absorbance at 400 to 412 nm of one or more standard samples having an sPLA₂ concentration of 50 ng/ml or greater;
wherein said subject is diagnosed as having a condition associated with elevated sPLA₂ levels if said test absorbance is at least 90% of said average standard absorbance.

49. The method of claim 48, wherein said one or more standard samples have an sPLA₂ concentration of 75 ng/ml or greater.

50. The method of claim 49, wherein said one or more standard samples have an sPLA₂ concentration of 100 ng/ml or greater.

51. The method of claim 48, wherein said biological fluid is selected from the group consisting of serum and urine.

52. The method of claim 48, wherein said incubation time in step (d) is 15 to 60 minutes.

53. The method of claim 52, wherein said incubation time in step (d) is 15 to 25 minutes.

54. The method of claim 48, wherein said development time in step (f) is 15 to 90 minutes.

55. The method of claim 54, wherein said development time in step (f) is 15 to 25 minutes.

56. The method of claim 54, wherein said development time in step (f) is 30 minutes to 90 minutes.

57. The method of claim 48, wherein said condition associated with elevated sPLA₂ levels is a condition associated with inflammation.

58. The method of claim 57, wherein said condition associated with inflammation is selected from the group consisting of atherosclerosis, coronary artery disease, multiple sclerosis, Alzheimer's disease, sickle cell disease, rheumatoid arthritis, and osteoarthritis.
